(19) United States
(12) Patent Application Publication
(10) Pub. No.: US 2007/0015737 A1
(43) Pub. Date: Jan. 18, 2007

(54) Compounds for inhibiting diseases and preparing cells for transplantation

(75) Inventors: Anne Clark, Oxon (GB); Paul Fraser, Toronto (CA); Bruce Verchere, Vancouver (CA); Ajay Gupta, Pointe-Claire (CA); David Migneault, Laval (CA); Walter Szarek, Kingston (CA)

Correspondence Address: LAHIVE & COCKFIELD, 28 STATE STREET, BOSTON, MA, 02109, US

(73) Assignees: Neurochem (International) Limited, Lausanne (CH); Isis Innovation Limited, Oxford (GB); Queen's University at Kingston, Kingston (CA); The University of British Columbia, Vancouver (CA); The Governing Counsel of The University of Toronto

(21) Appl. No.: 11/265,537

(22) Filed: Nov. 2, 2005

Related U.S. Application Data
(63) Continuation of application No. 10/030,350, filed on Nov. 8, 2002, now abandoned.
(60) Provisional application No. 60/142,907, filed on Jul. 9, 1999.
(60) Provisional application No. 60/142,953, filed on Jul. 12, 1999.

(30) Foreign Application Priority Data
Jul. 9, 1999 (GB) 9916214.1
Jul. 12, 1999 (GB) 9916315.6

(51) Int. Cl. A61K 31/66; G01N 33/567; A61K 31/44; A61K 31/47; A61K 31/445; A61K 31/185; C12N 5/08; A61K 31/19; G01N 33/53

(52) U.S. Cl. 514/114; 514/307; 514/553; 514/311; 514/317; 514/277; 514/574; 435/366; 435/7.2

(57) ABSTRACT

Methods and compositions which are useful in the treatment of amyloidosis. In particular, methods and compositions are provided for inhibiting, preventing and treating amyloid depositions, e.g. in pancreatic islets, wherein the amyloidotic deposits are islet amyloid polypeptide (IAPP)-associated amyloid deposition or deposits. The methods of the invention involved administering to a subject a therapeutic compound which inhibits IAPP-associated amyloid deposits. Accordingly, the compositions and method of the invention are useful for inhibiting IAPP-associated amyloidosis in disorders in which such amyloid deposition occurs, such an diabetes. The invention also provides a process for the preparation of cells suitable for transplantation into a mammal, which cells are capable of forming fibrils, said process comprising contacting the cells with an inhibitor of fibril formation. In particular the process prepares cells for use in a method of treating diabetes. Also provided are a culture medium comprising the inhibitor and cells for transplantation.

### FIELD OF THE INVENTION

The invention relates to compounds for inhibiting amyloid deposits in vivo, cells for transplantation, a process for preparing the cells and a medium for culturing the cells. In particular the invention relates to the inhibition of islet amyloid polypeptide (IAPP) deposition in vivo and a process for the preparation of islet cells for transplantation into patients with diabetes.

### BACKGROUND TO THE INVENTION

Amyloidosis refers to a pathological condition characterized by the presence of amyloid. Amyloid is a generic term referring to a group of diverse but specific intra- and extracellular protein deposits which are associated with a number of different diseases. The protein deposits comprise largely insoluble fibrillar material. The deposition of normally soluble proteins in this insoluble form is believed to lead to tissue malfunction and cell death.

Though diverse in their occurrence, all amyloid deposits have common morphological properties, including that they stain with specific dyes (e.g. Congo red), and have a characteristic birefringent appearance (sometimes characterized as “red-green”) in polarized light after staining. They also share common ultrastructural features and common x-ray diffraction and infrared spectra. At least some of the protein in the deposits is in the form of fibrils. Many different proteins are known to form fibrils. Such fibrils consist of long cylindrical structures in which the proteins comprise β-sheets that propagate in the direction of the fibril twisting around each other.

Amyloidosis can be classified clinically as primary, secondary, familial and/or isolated. Isolated forms of amyloidosis are those that tend to involve a single organ system compared to systemic amyloidosis involving many organs and tissues. Different amyloids are characterized by the type of protein present in the deposit. For example, neurodegenerative diseases such as scrapie, bovine spongiform encephalitis, Creutzfeldt-Jakob disease and the like are characterized by the appearance and accumulation of a protease-resistant form of a prion protein (referred to as AScr or PrP-27) in the central nervous system. Similarly, Alzheimer's disease, another neurodegenerative disorder, is characterized by congophilic angiopathy and neuritic plaques which have the characteristics of amyloid. In this localised form of amyloid the plaque and blood vessel amyloid is formed by the Alzheimer beta protein. Other diseases, such as complications of long-term hemodialysis and sequelae of long-standing inflammation or plasma cell dyscrasias are characterized by the accumulation of amyloid systemically. In each of these cases, a different amyloidogenic protein is involved in amyloid deposition.

Islet amyloid polypeptide (IAPP), also known as “amylin”, is known to be capable of forming fibrils which are deposited in the pancreatic islets of patients with Type II diabetes, forming deposits. Once these amyloid deposits have formed, there is no known therapy or treatment which significantly prevents, reduces or clears the deposits in situ. The inventors have now identified compounds for this purpose.

With regard to another aspect of the invention, diseases caused by the death or malfunctioning of a particular type or types of cells can be treated by transplanting into the patient healthy cells of the relevant type of cell. Often these cells are cultured in vitro prior to transplantation to increase their numbers, to allow them to recover after the isolation procedure or to reduce their immunogenicity. However, in many instances the transplants are unsuccessful, due to the death of the transplanted cells. The inventors have now also found that culturing of cells can lead to the formation of fibrils from endogenous proteins. Such fibrils are likely to continue to grow after the cells are transplanted and cause death or dysfunction of the cells. The inventors have shown that inhibitors of fibril formation can be used to inhibit the formation of fibrils in vitro.

### SUMMARY OF THE INVENTION

The inventors have now identified compounds that can be used to inhibit, reduce or disrupt amyloid deposits in vivo. In particular the compounds can be used against amyloid deposits of IAPP in vivo. Thus the invention provides methods and compositions which are useful in the treatment of amyloidosis. In particular, methods and compositions are disclosed for inhibiting, preventing and treating amyloid deposition, for example, in pancreatic islets wherein the amyloidotic deposits to be treated are, in an embodiment, islet amyloid polypeptide (IAPP)-associated amyloid deposits, e.g., having at least some β-sheet structure. The methods of the invention involve administering to a subject a therapeutic compound which inhibits, reduces or disrupts amyloid deposits, e.g., IAPP-associated amyloid deposits. Accordingly, the compositions and methods of the invention are useful for inhibiting amyloidosis in disorders in which such amyloid deposition occurs, such as diabetes.

In one embodiment, a method for inhibiting amyloid deposition, particularly IAPP-associated amyloid deposition, in a subject is provided, wherein an effective amount of an IAPP fibril inhibiting compound, or a pharmaceutically acceptable salt thereof, is administered to the subject such that said IAPP-associated amyloid deposition is inhibited. Such compounds include those of the following general formula (a): 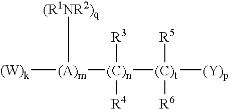
wherein k, m, t, p and q are independently 0 or 1; n is an integer from 0 to 3; C is a carbon; N is a nitrogen; W is hydrogen or an anionic group at physiological pH; Y is an anionic group at physiological pH; R¹ and R² are independently hydrogen, alkyl, an anionic group at physiological pH, or R¹ and R², taken together with the nitrogen to which they are attached, may form an unsubstituted or substituted heterocycle having from 3 to 7 atoms in the heterocyclic ring; R³ is hydrogen halogen, thiol or hydroxyl; R⁴, R¹ and R⁶ are independently hydrogen or halogen; and A is hydrogen or C₁ to C₆ alkyl; or a pharmaceutically acceptable ester, acid or salt thereof.

Preferred therapeutic compounds include 3-(3-hydroxy-1-propyl)amino-1-propanesulfonic acid; DL-2-amino-5-phosphovaleric acid; 4-Phenyl-1-(3′-sulfopropyl)-1,2,3,6-tetrahydropyridine; cyclohexylsulfamic acid; O-phospho-L-serine; hexafluoroglutaric acid; 8-methoxyquinoline-5-sulfonic acid; 3-amino-2-hydroxy-1-propanesulfonic acid; and 3-dimethylamino-1-propanesulfonic acid, and pharmaceutically acceptable esters, acids or salts thereof.

In another embodiment a method for inhibiting amyloid deposition, particularly IAPP-associated amyloid deposition, in a subject is provided, wherein an effective amount of an IAPP fibril inhibiting compound, or a pharmaceutically acceptable ester, acid or salt thereof, is administered to the subject such that said IAPP-associated amyloid deposition is inhibited. Such compounds include those of the following general formula (b): 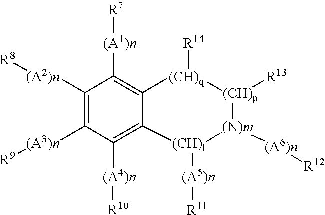
wherein C is a carbon; N is a nitrogen; H is a hydrogen; A¹, A², A³, A⁴, A⁵ and A⁶ are independently alkyl, O, S, or —NH; m and n (for each individual A group) are independently 0 or 1; p, q and l are independently 0, 1, or 2; R⁷, R⁸, R⁹, R¹⁰, R¹¹, R¹², and each R¹⁴ are independently hydrogen, alkyl, alicyclyl, heterocycyl or aryl, each R¹³ is independently hydrogen, alkyl, alicyclyl, heterocycyl, aryl or an anionic group, and adjacent R groups (e.g., R⁷ and RR) may form an unsubstituted or substituted cyclic or heterocyclic ring.

Preferred therapeutic compounds include 1,2,3,4-tetrahydroisoquinoline. In another embodiment the invention relates to a method for reducing IAPP-associated amyloid deposits in a subject having IAPP-associated amyloid deposits, the method comprising administering to a subject an effective amount of an IAPP fibril inhibiting compound, or a pharmaceutically acceptable salt thereof, such that IAPP-associated amyloid deposits are reduced.

The therapeutic compounds of the invention are administered to a subject by a route which is effective for inhibiting IAPP-associated amyloid deposition. Suitable routes of administration include oral, transdermal, subcutaneous, sublingual, buccal, intravenous and intraperitoneal injection. The therapeutic compounds can be administered with a pharmaceutically acceptable vehicle.

The invention further provides pharmaceutical compositions for treating amyloidosis. The pharmaceutical compositions include a therapeutic compound of the invention in an amount effective to inhibit IAPP-associated amyloid deposition, and a pharmaceutically acceptable vehicle.

The inventors have also found that the culturing of cells in vitro can lead to the formation of fibrils from endogenous proteins. Since the process is progressive, the fibrils are likely to continue to grow after the cells are transplanted and cause the death or dysfunction of the cells. This may occur even when the cells are from a healthy donor and when the patient receiving the transplant does not have a disease that is characterised by the presence of fibrils. The inventors have shown that the culturing of islet cells from a non-diabetic donor for the purpose of transplantation into a patient with type I diabetes leads to the formation of fibrils in cell clusters in vitro. They have also shown that inhibitors of fibril formation can be used to reduce the formation of fibrils in vitro.

Thus the invention also provides a process for the preparation of cells suitable for transplantation into a mammal, which cells are capable of forming fibrils, said process comprising contacting the cells with an inhibitor of fibril formation. Any inhibitor of fibril formation may be used, including any such compounds mentioned herein.

The invention also provides a culture medium or culture medium pre-mix that comprises an inhibitor of the invention. The invention further provides ex vivo cells made by the process of the invention. The invention provides the ex vivo cells for use in a method of treatment of the human or animal body by therapy. In particular the invention provides ex vivo cells of the invention which are islet cells for use in a method of treating diabetes.

### BRIEF DESCRIPTION OF THE DRAWINGS

FIG. 1 shows the effect of candidate substances on the proportion of non-fibrillar IAPP in a mixture of fibrils and non-fibrillar IAPP at time 0 and after incubation over a period of 24 h. The unshaded columns show results at time 0 and the shaded columns show results at time 24 h. The vertical axis shows the proportion of non-fibrillar IAPP in the supernatent (as a percentage of control). Changes in the direction of the arrow represent a relative increase in non-fibrillar IAPP (decrease in fibrils).

FIG. 2 shows the effect of polyvinylsulfonate (A) and compound vi (C), compared to IAPP alone (B), on formation of fibrils. The vertical axis shows fluorescence units. The horizontal axis shows time in hours.

FIG. 3 shows the effects of compounds i, iii, x and iv on molecular conformation of the peptide as measured by circular dichroic spectroscopy at time 0 (left) and time 24 h (right). The horizontal axis shows the wave length.

FIGS. 4a and 4b show respectively isolated human and mouse islet cells which have been cultured for 6 days in glucose and RPM 1. Amyloid fibrils can be seen between the cells (as shown by arrows). The dots show fibrils which have been immunogold labelled for IAPP.

FIG. 5 shows the fibrils formed by IAPP alone or in the presence of candidate substances.

FIG. 6 shows mean islet survival in the presence of the compounds. (A) represents guandinoethanosulfonic acid.

### DETAILED DESCRIPTION OF THE INVENTION

The present invention will be more fully illustrated by reference to the definitions set forth below.

“Amyloid” includes IAPP-associated amyloid, including, but not limited to, β-sheet amyloid assembled substantially from IAPP subunits. “Inhibition” of amyloid deposition includes preventing or stopping of IAPP-associated amyloid formation, inhibiting or slowing down of further IAPP-associated amyloid deposition in a subject with ongoing amyloidosis, e.g., already having amyloid deposits, and reducing or reversing IAPP-associated amyloid deposits in a subject with ongoing amyloidosis. Inhibition of amyloid deposition is determined relative to an untreated subject, or relative to the treated subject prior to treatment, or, e.g., determined by clinically measurable improvement in pancreatic function in a diabetic patient.

Pharmaceutically acceptable salts of the therapeutic compound, where applicable, are within the scope of the invention, e.g., alkali metal, alkaline earth metal, higher valency cation (e.g., aluminum salt), polycationic counter ion or ammonium salts. Where a compound is anionic, a preferred pharmaceutically acceptable salt is a sodium salt. Other salts are also contemplated, e.g., HCl, citric acid, tartaric acid salts, within their pharmaceutically acceptable ranges.

The therapeutic compound of the invention can be administered in a pharmaceutically acceptable vehicle. As used herein “pharmaceutically acceptable vehicle” includes any and all solvents, dispersion media, coatings, antibacterial and antifungal agents, isotonic and absorption delaying agents, and the like which are compatible with the activity of the compound and are physiologically acceptable to the subject. An example of a pharmaceutically acceptable vehicle is buffered normal saline (0.15 molar NaCl). Except insofar as any conventional media or agent is incompatible with the therapeutic compound, use thereof in the compositions suitable for pharmaceutical administration is contemplated. Supplementary active compounds can also be incorporated into the compositions.

An “anionic group,” as used herein, refers to a group that is negatively charged at physiological pH (for example at pH 6.6) or at a pH at which mammalian cells can be cultured (for example any such pH ranges mentioned herein). Preferred anionic groups include carboxylate, sulfate, sulfonate, sulfinate, sulfamate, tetrazolyl, phosphate, phosphonate, phosphinate, and phosphorothioate or functional equivalents thereof. “Functional equivalents” of anionic groups include bioisosteres, e.g., bioisosteres of a carboxylate group. Bioisosteres encompass both classical bioisosteric equivalents and nonclassical bioisosteric equivalents. Classical and non-classical bioisosteres are known in the art (see, e.g., Silverman, R. B. The Organic Chemistry of Drug Design and Drug Action, Academic Press, Inc.: San Diego, Calif., 1992, pp. 19-23). A particularly preferred anionic group is a carboxylate.

The term “alkyl” includes saturated aliphatic groups, including straight-chain alkyl groups, branched-chain alkyl groups, cycloalkyl(alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably has 20 or fewer carbon atoms in the backbone. Likewise, cycloalkyls may have from 4-10 carbon atoms in their ring structure, more preferably have 5, 6 or 7 carbons in the ring structure.

Moreover, the term alkyl includes both “unsubstituted alkyls” and “substituted alkyls”, the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “aralkyl” moiety is an alkyl substituted with an aryl (e.g., phenylmethyl(benzyl)).

The term “aryl” herein includes 5- and 6-membered single-ring aromatic groups that may include from zero to four heteroatoms, for example, benzene, pyrrole, furan, thiophene, imidazole, oxazole, thiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles”, “heteroaryls” or “heteroaromatics”. The aromatic ring can be substituted at one or more ring positions with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety. Aryl groups can also be fused or bridged with alicyclic or heterocyclic rings which are not aromatic so as to form a polycycle (e.g., tetralin).

The terms “alkenyl” and “alkynyl” include unsaturated aliphatic groups analogous in length and possible substitution to the alkyls described above, but that contain at least one double or triple bond respectively.

Unless the number of carbons is otherwise specified, “lower alkyl” means an alkyl group, as defined above, but having from one to ten carbons, more preferably from one to six carbon atoms in its backbone structure. Likewise, “lower alkenyl” and “lower alkynyl” have similar chain lengths. Preferred alkyl groups are lower alkyls.

The terms “heterocyclyl” or “heterocyclic group” include 3- to 10-membered ring structures, more preferably 4- to 7-membered rings, which ring structures include one to four heteroatoms. Heterocyclyl groups include pyrrolidine, oxolane, thiolane, piperidine, piperazine, morpholine, lactones, lactams such as azetidinones and pyrrolidinones, sultams, sultones, and the like. The heterocyclic ring can be substituted at one or more positions with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety.

The terms “polycyclyl” or “polycyclic group” include two or more cyclic rings (e.g., cycloalkyls, cycloalkenyls, cycloalkvnyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety.

The term “heteroatom” includes an atom of any element other than carbon or hydrogen. Preferred heteroatoms are nitrogen, oxygen, sulfur and phosphorus.

The term “aryl aldehyde,” as used herein, includes compounds represented by the formula Ar—C(O)H, in which Ar is an aryl moiety (as described above) and —C(O)H is a formyl or aldehydo group.

It will be noted that the structure of some of the compounds of this invention includes asymmetric carbon atoms. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis. Furthermore, alkenes can include either the E- or Z-geometry, where appropriate.

The present methods and compositions, in embodiments, inhibit, prevent and treat amyloid deposition in pancreatic islets wherein the amyloidotic deposits to be treated are islet amyloid polypeptide (IAPP)-associated amyloid deposits, e.g., having at least some β-sheet structure. The methods of the invention include administering to a subject a therapeutic compound which inhibits, reduces or disrupts IAPP-associated amyloid deposits. Accordingly, the compositions and methods of the invention are useful for inhibiting amyloidosis in disorders in which such amyloid deposition occurs, such as diabetes.

In one embodiment, a method for inhibiting IAPP-associated amyloid deposition in a subject is provided, wherein an effective amount of an IAPP fibril inhibiting compound, or a pharmaceutically acceptable salt thereof, is administered to the subject such that said IAPP-associated amyloid deposition is inhibited. Such compounds include those of the following general formula (a) 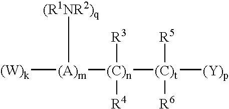
wherein k, m, t, p and q are independently 0 or 1; n is an integer from 0 to 3; C is a carbon, N is a nitrogen; W is hydrogen or an anionic group at physiological pH; Y is an anionic group at physiological pH; R¹ and R² are independently hydrogen, alkyl, an anionic group at physiological pH, or R¹ and R², taken together with the nitrogen to which they are attached, may form an unsubstituted or substituted heterocycle having from 3 to 7 atoms in the heterocyclic ring; R³ is hydrogen, halogen, thiol or hydroxyl; R⁴, R⁵, and R⁶ are independently hydrogen or halogen; and A is hydrogen or C₁ to C₆ alkyl; or a pharmaceutically acceptable salt thereof.

In an embodiment, W is preferably —COOH; Y is preferably —COOH, —SO₃H, —PO₃H₂ or —OP(O)(OH)₂; R¹ is preferably H, Me or hydroxypropyl; R² is preferably H, Me or —SO₃H; R³ is preferably H, F, or OH; when R¹ and R², taken together with the nitrogen to which they are attached, form an unsubstituted or substituted heterocycle, preferred groups include 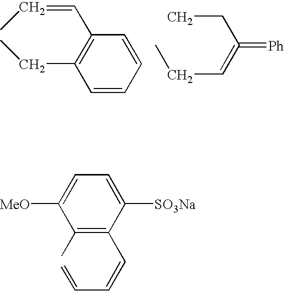
R⁴, R⁵ and R⁶ are preferably H or F; A is preferably H, CH, CF₂ or alkyl which may be substituted or unsubstituted, straight, branched or cyclic, e.g., cyclohexyl.

Preferred therapeutic compounds include 3-(3-hydroxy-1-propyl)amino-1-propanesulfonic acid; DL-2-amino-5-phosphovaleric acid; 4-Phenyl-1-(3′-sulfopropyl)-1,2,3,6-tetrahydropyridine; cyclohexylsulfamic acid; O-phospho-L-serine; hexafluoroglutaric acid; 8-methoxyquinoline-5-sulfonic acid; 3-amino-2-hydroxy-1-propanesulfonic acid; and 3-dimethylamino-1-propanesulfonic acid, and pharmaceutically acceptable salts thereof.

In another embodiment a method for inhibiting IAPP-associated amyloid deposition in a subject is provided, wherein an effective amount of an IAPP fibril inhibiting compound, or a pharmaceutically acceptable salt thereof, is administered to the subject such that said IAPP-associated amyloid deposition is inhibited. Such compounds include those of the following general formula (b) 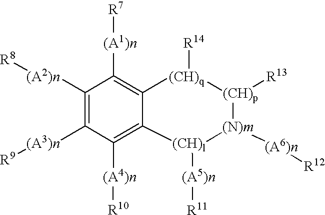
wherein C is a carbon; N is a nitrogen; H is a hydrogen; A¹, A², A³, A⁴, A¹ and A⁶ are independently alkyl, O, S, or —NH; m and n (for each individual A group) are independently 0 or 1; p, q and l are independently 0, 1, or 2; R⁷, R⁸, R⁹, R¹⁰, R¹¹, R¹², and each R¹⁴ are independently hydrogen, alkyl, alicyclyl, heterocycyl or aryl, each R¹³ is independently hydrogen, alkyl, alicyclyl, heterocycyl, aryl or an anionic group, and adjacent R groups (e.g., R⁷ and R⁸) may form an unsubstituted or substituted cyclic or heterocyclic ring.

Preferred therapeutic compounds include 1,2,3,4-tetrahydroisoquinoline.

A further aspect of the invention includes pharmaceutical compositions for treating amyloidosis. The therapeutic compounds in the methods of the invention, as described hereinbefore, can be incorporated into a pharmaceutical composition in an amount effective to inhibit amyloidosis or reduce amyloid deposits, in a pharmaceutically acceptable vehicle.

In the methods of the invention, amyloid deposition in a subject is inhibited by administering a therapeutic compound of the invention to the subject. The term subject includes living organisms in which amyloidosis can occur. Examples of subjects include humans, apes, monkeys, cows, sheep, goats, dogs, cats, mice, rats, and transgenic species thereof, as well as cells therefrom, e.g., islet cells, in culture. Administration of the compositions of the present invention to a subject to be treated can be carried out using known procedures, at dosages and for periods of time effective to inhibit amyloid deposition or reduce amyloid deposits in the subject. An effective amount of the therapeutic compound necessary to achieve a therapeutic effect may vary according to factors such as the amount of amyloid already deposited at the clinical site in the subject, the age, sex, and weight of the subject, and the ability of the therapeutic compound to inhibit amyloid deposition or reduce amyloid deposits in the subject. Dosage regimens can be adjusted to provide the optimum therapeutic response. For example, several divided doses may be administered daily or the dose may be proportionally reduced as indicated by the exigencies of the therapeutic situation.

The active compound may be administered by routes such as oral, sublingual, buccal, transdermal, subcutaneous, intravenous, and intraperitoneal administration. Depending on the route of administration, the active compound may be coated in a material to protect the compound from the action of acids, enzymes and other natural conditions which may inactivate the compound.

The compounds of the invention can be formulated to ensure proper distribution in vivo. For example, the therapeutic compounds of the invention can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs (“targeting moieties”), thus providing targeted drug delivery (see, e.g., V. V. Ranade (1989). J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995)FEBS Lett. 357:140; M. Owais et al. (1995)Antimicrob. Agents Chemother. 39:180); surfactant protein A receptor (Briscoe et al. (1995) Am. J. Physiol. 1233:134); gp120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273. In a preferred embodiment, the therapeutic compounds of the invention are formulated in liposomes; in a more preferred embodiment, the liposomes include a targeting moiety.

To administer the therapeutic compound by other than parenteral administration, it may be necessary to coat the compound with, or co-administer the compound with, a material to prevent its inactivation. For example, the therapeutic compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., (1984) J. Neuroimmunol. 7:27).

The therapeutic compound may also be administered parenterally, sublingually, buccally, intraperitoneally, intraspinally, or intracerebrally. Dispersions can be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The vehicle can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filter sterilization. Generally, dispersions are prepared by incorporating the therapeutic compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yield a powder of the active ingredient (i.e., the therapeutic compound) plus any additional desired ingredient from a previously sterile-filtered solution thereof.

The therapeutic compound can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, sublingual/buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of amyloid deposition in subjects.

Active compounds are administered at a therapeutically effective dosage sufficient to inhibit amyloid deposition in a subject. A “therapeutically effective dosage” preferably inhibits amyloid deposition and/or reduces amyloid deposits by at least about 20%, more preferably by at least about 40%, even more preferably by at least about 60%, and still more preferably by at least about 80% relative to untreated subjects or to the same subject prior to treatment.

The ability of a compound to inhibit amyloid deposition or reduce amyloid deposits can be evaluated in an animal model system that may be predictive of efficacy in inhibiting amyloid deposition or reducing amyloid deposits in human diseases. The ability of a compound to inhibit amyloid deposition can also be evaluated by examining the ability of the compound to inhibit amyloid deposition in vitro or ex vivo, e.g., using an ELISA assay. The effect of a compound on the secondary structure of the amyloid can be further be determined by circular dichroism (CD) or infrared (IR) spectroscopy.

CD and IR spectroscopy are particularly useful techniques because the information obtained is a direct measure of the ability of a test compound to prevent or reverse amyloidosis, by determining the structural effect of a compound on amyloid protein folding and/or fibril formation. This contrasts with previously known methods which measure cellular trafficking of amyloid protein precursors or interactions between amyloid and extracellular matrix proteins, providing only indirect evidence of potential amyloid-inhibiting activity. It should further be noted that CD and IR spectroscopy can also detect compounds which cause an increase in, e.g., β-sheet folding of amyloid protein, and thereby stabilize the formation of amyloid fibrils.

The deposition of amyloid is a multi-stage process. Accordingly, an agent useful for treating amyloidosis has many potential modes of action. An agent which inhibits amyloid deposition could act in one or more of the following ways, which are shown by way of illustration and not limitation:
1. Inhibition or delay of protein folding in solution
2. Inhibition or delay of aggregation of amyloid peptides into fibrils and/or deposits
3. Disruption/dissolution/modification of amyloid fibrils and/or deposits Categories 1 and 2 correspond to prevention of the formation of amyloid deposits (slowing down or halting amyloid deposition), and category 3 corresponds to removal or modification of deposits already formed (removal or reduction of existing amyloid deposits).

In another aspect the process of the invention aims to reduce the amount of amyloid deposits that are present in a cell preparation before transplantation. As mentioned amyloid deposits will comprise at least some protein present in the form of fibrils.

Such fibrils typically have an ordered and repeating structure created by the regular assembly of the protein components. Typically a fibril is straight and unbranched. It is generally insoluble in the cytoplasm or in extracellular compartments. The fibril may or may not be insoluble in distilled water, or organic solvents, such as hexafluoroisopropanol or trifluoroethanol. It typically has a diameter of 5 to 20 nm, for example 7 to 15 or 10 to 12 nm. The protein in the fibril generally forms one, two or more β-strands which are typically oriented substantially perpendicular to the long axis of the fibril and may form β-sheets that propagate substantially in the direction of the fibril twisting around each other. Fibrils are generally in the form of small linear aggregates of molecules in β-sheet construction or of filamentous structure of varying extended lengths.

Typically the protein that forms the fibril has at least 30%, such as at least 50 or 70% of its native structure as β-sheet. In the case of certain fibrils the native form of the protein (i.e. the soluble non-fibril form) has an α-helical region, which may in all or part be converted to a β-sheet structure in the fibril. The protein is typically a secreted extracellular protein, but may be an intracellular protein. The protein may be the wild-type or an alternative form, such as a mutated form. The alternative form can be a truncated form of the wild-type protein.

The protein is typically IAPP, Aβ peptide (involved in Alzheimer's disease), prion protein, immunoglobulin light chain, amyloid A protein, transthyretin, cystatin, β2-microglobulin, apolipoprotein A-1, gelsolin, calcitonin, atrial natriuretic factor, lysozyme variants, insulin, or fibrinogen.

The protein may be one which has sequence or structural homology with any of these particular proteins. Preferably the protein has sequence or structural homology with IAPP. The protein may be one which does not contain any tryptophan residues in its sequence.

The inhibitor of fibril formation is able to reduce the amount of fibril formation that occurs in conditions in which fibril formation would occur. Thus an inhibitor can be identified in an assay by contacting a candidate substance with a protein that forms fibrils under conditions in which fibril formation would occur and determining whether fibril formation is inhibited by the substance. In one embodiment the inhibitor may interact with preformed fibrils to modulate their architecture resulting in the breaking of the fibrils into monomeric or small oligomeric peptide components. The protein may be any of the proteins mentioned herein.

In the in vitro assay, changes in the proportion of monomeric/small oligomeric protein components in a mixture of protein and fibrils can be measured by assaying the non-fibrillar components. The effects of candidate substances on fibril formation can also be measured using thioflavine T spectroscopy or circular dichroic spectroscopy. Circular dichroic spectroscopy indicates the effect of the candidate substances on the molecular conformation of the soluble or other forms of non-fibrillar peptide. In particular, conversion of the molecule to β-conformation which indicates β-sheet formation.

In the assay the inhibitor typically inhibits fibril formation by at least 10%, at least 20%, at least 30%, at least 40% at least 50%, at least 60%, at least 70%, at least 80%, at least 90%, at least 95%, at least 99% or at least 99.9% at a concentration of the inhibitor of 10 ng ml⁻¹, 100 ng ml⁻¹, 1 μg ml⁻¹, 10 μg ml⁻¹, 100 μg ml⁻¹, 500 μg ml⁻¹, 1 mg ml⁻¹, 10 mg ml⁻¹ or 100 mg ml⁻¹, or a molarity of inhibitor of 100 nM, 1 μM, 10 μM, 100 μM, 1 mM, 10 mM or 100 mM. In one embodiment such effects are measured over 24 hours in an assay in which the concentration of the monomeric protein is 20 μM. The percentage inhibition represents the percentage decrease in amount of fibril formation in a comparison of assays in the presence and absence of the test substance. Any combination of the above mentioned degrees of percentage inhibition and concentration of inhibitor may be used to define an inhibitor of the invention, with greater inhibition at lower concentrations being preferred.

An inhibitor of IAPP fibril formation typically causes any of the above percentage inhibition at any of the above concentrations or molarities when contacted with an 80 μg/ml solution of human IAPP in distilled water, at 25° C., over 24 hours.

The inhibitor is typically non-toxic towards the cells (e.g. in culture), for example at any of the concentrations mentioned above. The inhibitor may be non-toxic towards any of the mammals mentioned herein, and thus may be pharmaceutically acceptable. The inhibitor may or may not be able to enter the cells, typically by diffusing across the cell membrane.

The inhibitor may prevent the neoformation and/or growth of fibrils and/or may breakdown any preformed fibrils which are present. The inhibitor typically binds to the monomeric form of the protein and prevents it oligomerising to form the fibril. The inhibitor may bind to the multimeric form to prevent further protein binding and/or modulate the structure of the multimeric form to cause breakdown of the multimeric form into its component peptide fragments. Such a binding may be reversible or non-reversible. The binding may cause a change in the structure of the monomeric or multimeric form of the protein and/or a change in the structure of the inhibitor. In one embodiment the surface of the inhibitor mimics the part of the surface of the monomeric form which will bind the multimeric form.

The inhibitor may be congo red (e.g as described in WO 94/01116), an acridinone or related molecule (e.g. as described in WO 97/16191), a naphthylazo compound (e.g. as described in WO 97/16194), hexadecyl-N-methylpiperidinium bromide (J. Biol. Chem. (1986) 271 (8), 4086-4092), or a saccharide or saccharide composition (e.g. as described in WO 99/0999). The inhibitor may be a peptide or a substituted peptide as described in J. Am. Chem. Soc. (1998) 120, 7655-7656. The inhibitor may be an organic solvent (e.g. dimethylsulphoxide or polyethyleneglycol).

The inhibitor may be of the formula (a) or (b), including any of the specific embodiments or specific compounds covered by (a) or (b) which are discussed above.

Preferably the inhibitor is any one of compounds (i) to (x) below or a pharmaceutically acceptable salt thereof:
(i) 3-(3-hydroxy-1-propyl)amino-1-propanesulfonic acid
   HOCH₂CH₂CH₂NHCH₂CH₂CH₂SO₃H,
(ii) DL-2-amino-5-phosphovaleric acid 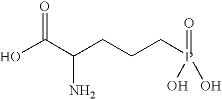
(iii) 1,2,3,4-tetrahydroisoquinoline 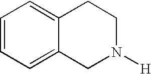
(iv) Cyclohexylsulfamic acid 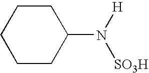
(v) O-Phospho-L-serine 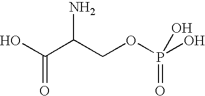
(vi) Hexafluoroglutaric acid 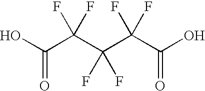
(vii) 8-methoxyquinoline-5-sulfonic acid 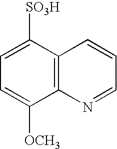
(viii) 4-phenyl-1-(3′ sulfopropyl)-1,2,3,6-tetrahydropyridine 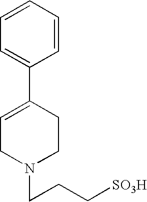
(ix) 3-amino-2-hydroxy-1-propanesulfonic acid 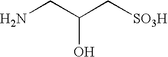
(x) 3-dimethylamino-1-propanesulfonic acid
   Me₂NCH₂CH₂CH₂SO₃H

| | Form of | | |
|---|---|---|---|
| Compound No. | Compound | CAS Number | Supplier |
| i | | 58431-88-2 | Neurochem Inc. |
| ii | | 76326-31-3 | Sigma-Aldrich |
| iii | Hydrochloride | 14099-81-1 | Sigma-Aldrich |
| iv | Sodium salt | 139-05-9 | Sigma-Aldrich |
| v | | 407-41-0 | Sigma-Aldrich |
| vi | | 376-73-8 | Sigma-Aldrich |
| vii | | 40712-20-7 | Sodium salt available |
| | | | from Neurochem Inc. |
| viii | Sodium salt | | Neurochem Inc. |
| ix | | 7013-33-4 | Neurochem Inc. |
| x | | 29777-99-9 | Neurochem Inc. |

In the discussion below the term ‘specific inhibitor’ includes any of the compounds discussed herein, such as those described by either of the general formulae (a) or (b) or their salts (including (i) to (x) above as well as their salts discussed above). The inhibitor of the invention may be structurally and/or functionally equivalent to any of the specific inhibitors. Thus the inhibitor of the invention may be capable of competing with any of the specific inhibitors to bind the monomeric or multimeric form of the protein. Thus the inhibitor may bind the monomeric or multimeric form at the same place as any of the specific inhibitors. The inhibitor may mimic the surface of any of the specific inhibitors. Thus the inhibitor of the invention may bind to an antibody that binds to the specific inhibitors, and thus may be capable of inhibiting the binding of the antibody to the specific inhibitor. Such an antibody can be made by known methods, including administering the specific inhibitor to an animal in association with a carrier to make it more immunogenic. The inhibitor may mimic the shape, size, flexibility or electronic configuration of any of the specific inhibitors. It is typically a derivative of any of the specific inhibitors.

The antibody may be used to identify inhibitors from libraries of compounds, such as combinatorial libraries. Alternatively inhibitors which mimic the specific inhibitors may be designed computationally and made by synthetic chemistry techniques.

The cells of the invention are generally going to be transplanted into a patient suffering from a disease. The disease may or may not be a disease in which fibrils are present in the patient. In the case of diseases in which fibrils are present in the patient the fibrils may cause the disease, or at least some of the symptoms of the disease. The fibrils may be formed because of the disease, and may exacerbate the symptoms of the disease. The disease may be type I or type II diabetes, Alzheimer's disease, a spongiform encephalopathy, primary or secondary systemic amyloidosis, familial amyloidotic polyneuropathy, senile systemic amyloidosis, hereditary cerebral amyloid angiopathy, haemodialysis-related amyloidosis, Finnish hereditary amyloidosis, medullary carcinoma of the thyroid, atrial amyloidosis, lysozyme amyloidosis, or fibrinogen α-chain amyloidosis.

In the disease fibrils may form in a particular tissue, and thus may affect a particular type or types of cells. The fibrils may be made of a protein produced by such cells. Alternatively the fibrils may be made of a protein which is produced in a different type of cell or tissue. The fibrils may be located in an intracellular compartment of the cells, such as in the cytoplasm or cytoplasmic organelles. Alternatively the fibrils are located in the extracellular compartment, for example in the proximity of or contacting the cells, which have produced the protein from which the fibrils are made.

Alternatively in the disease the cells may be affected by other factors which are not connected with fibrils, for example by an autoimmune attack or pathogenic infection, such as a bacterial or viral infection. The disease may be type I diabetes. Such cells being affected in the disease may be the same as or different from the cells discussed herein. The cells of the invention are capable of forming fibrils or susceptible to the deleterious effect of fibrils.

Typically fibrils damage cells by causing a decrease in the amount of substances produced (or secreted) by the cells, or kill the cells or induce apoptosis and cell death.

Thus the ex vivo cells of the invention are typically of the same or similar type as the cells which have been affected by the disease. The process of the invention is applicable to cells that can form fibrils after transplantation, which as discussed above typically also form fibrils when in culture. In one embodiment the ex vivo cells are in a preparation that comprises the inhibitor.

The cells are mammalian cells, such as human, primate, rodent, rabbit, ovine, porcine, bovine, feline or canine cells. The cells are typically cells that naturally express a protein that is capable of forming a fibril. In one embodiment the cells are endocrine cells. The cells may be islet, liver, muscle, kidney or neuronal cells. In one embodiment the cells are genetically modified, and are, for example, capable of expressing genes not naturally expressed by the cell. In one embodiment the cells are islet cells and the fibrils comprise human islet amyloid polypeptide.

The cells of the invention are typically from a donor who generally does not have the relevant disease. In one embodiment the cells are from the patient. The cells may be take from the patient to increase their numbers in vitro and/or the cells may be treated therapeutically in some manner before administering them back to the patient. For example the cells may be treated with agents, which act against (e.g. kill) pathogens.

The cells can be obtained from the donor or patient by standard techniques. Before being cultured the cells are generally further purified, for example using collagenase dissociation and/or density gradient centrifugation techniques or cell sorting techniques.

The cells are contacted with the inhibitor before transplantation. Generally the cells are contacted with the inhibitor when being cultured. Thus in the process the cells are cultured in the presence of the inhibitor. However, in one embodiment the cells are contacted with inhibitor after culturing, but before transplantation, for example in the case of inhibitors which cause breakdown of pre-existing fibrils. In one embodiment of the process the fibrils are initially present in the said cells prior to contacting with said inhibitor and said inhibitor causes breakdown of said fibrils. The inhibitor may cause breakdown of some or of all the fibrils present.

The cells are typically cultured in order allow them to recover after the isolation procedure, to increase their numbers before transplantation, to treat them therapeutically in some manner or to change the proportion of the different types of cells present in the culture. In order to achieve this last aim the cells may be cultured in conditions which allow the survival of certain types of cells over other types of cells. In one embodiment the cells are cultured in conditions which reduce the numbers of ‘passenger’ leukocytes in order to reduce the immunogenicity of the cells.

Typically cells are cultured for from 12 to 150 hours, for example from 24 to 100 hours, before transplantation. Generally the cells are cultured at from 20° C. to 45°, for example 30° C. to 40° C., preferably 35° C. to 37° C. Generally the pH of the culture is from 6.6 to 8.0, preferably 7 to 7.6 or 7.2 to 7.4. Thus the cells of the invention may be cells which have been cultured under such conditions and/or in the culture medium discussed below.

Generally the cells are cultured in the culture medium of the invention which comprises an inhibitor of the invention. Such a medium is capable of providing support for any of the cells of the invention. Thus the medium will provide substances to keep the cells alive, and may also allow growth and replication of the cells. The medium thus comprises nutrition for the cells. The nutrition will be in the form of an assimible carbon source, such as a carbohydrate source or amino acids. Thus the medium may comprise sugars, such as glucose, fructose, mannose or galactose or non-sugar carbohydrates, such as lactate or pyruvate. These may be present at from 1 to 40 mM, such as 10 to 30 mM. The medium may comprise an amino acid, such as arginine, cysteine, histidine, isoleucine, leucine, lysine, methionine, phenylalanine, threonine, tryptophan, tyrosine or valine.

The medium comprises water. The medium may comprise ions from inorganic salts, such as sodium, potassium, calcium, magnesium, iron, selenium, carbonate, phosphate or sulphate ions. The medium may comprise vitamins, such as nicotinamide. The medium may comprise xanthine.

The medium may comprise growth factors or hormones. The medium may comprise proteins, such as binding and transport proteins, for example transferrin or albumin. The medium may comprise lipids, insulin or ethanolamine.

Some of the above components may be provided in the medium by an extract from an animal or equivalent supplements. Thus the medium may comprise such an extract. The extract may be partially purified. The extract may be serum, such as foetal calf serum.

The medium may comprise antibiotics, such as penicillin.

The medium may comprise the constituents of the commercially available mediums which can be obtained from NBL™ or Gibco™, such as RPMI 1640™, Dulbecco's modified Eagle's medium™, Medium 199™, CMRL 1066™.

In the medium the inhibitor is typically present at a concentration of 1 to 10,000 μM, for example 10 to 1000 μM or 100 to 500 μM.

The culture medium pre-mix typically has the same constituents as the culture medium but contains less water, such as less than from 50%, 10%, 1% or 0.1% of the water present in the culture medium. The pre-mix may be in the form of a liquid, gel or powder. Typically the pre-mix can be converted to a culture medium by adding water.

The culture medium may comprise a precursor of the inhibitor which provides the inhibitor when contacted with any of the cells discussed above. The pre-mix may comprise a precursor which provides the inhibitor when contacted with water or with the cells. The term ‘inhibitor’ includes such precursors.

The culture of the invention comprises the cells of the invention and culture medium of the invention. The culture comprises at least 100 cells, such as at least 10³, 10⁵, 10⁷ or 10⁹ cells

The inhibitor may also be administered to a patient who has received a transplant of the cells of the invention. The inhibitor is administered to prevent damage in vivo to the transplanted cells by fibrils. Thus the invention provides an inhibitor for use in inhibiting fibril formation by, or breaking fibrils down in, a transplanted cell preparation.

The invention also provides a vessel for containing a culture of cells, which vessel is coated with the inhibitor. The vessel may be a Petri dish or a flask. The vessel may comprise glass or plastic. The inhibitor is generally present on the surface which will be in contact with the culture. The inhibitor is present in a form in which it is capable of being released into the culture when the culture comes into contact with it.

The invention also provides a kit for culturing cells comprising the culture medium or pre-mix of the invention or a vessel of the invention.

Antibodies that bind to the inhibitors of the invention may be used to screen for inhibitors based on their ability to bind the antibody. Typically such a screening is carried out on a library of candidate compounds. Thus the invention provides use of an antibody that binds an inhibitor of the invention, or of a fragment that retains the ability of said antibody to bind said inhibitor, to identify a compound that can be used to prepare cells for transplantation in the process of the invention.

Inhibitors can also be identified based on their ability to inhibit the formation of fibrils or breakdown fibrils in a cell preparation that comprises any of the types of cells mentioned herein which can be treated in the process of the invention. Thus the invention provides a method of identifying an inhibitor that can be used to prepare cells for transplantation in a process of the invention, comprising contacting a candidate substance with a mammalian cell and determining whether the candidate substance inhibits the formation of fibrils or causes the breakdown of fibrils, (i) the inhibition of formation of fibrils or (ii) the breakdown of fibrils, indicating that the substance is an inhibitor that can be used in said process. The cell is typically a human islet cell.

The inhibitor may also identified based on its ability to breakdown fibrils or to inhibit the formation of fibrils. Thus the invention provides a method of identifying an inhibitor that can be used to prepare cells for transplantation in a process of the invention, comprising contacting a candidate substance with a protein capable of forming fibrils, or with a fibril, and determining whether the substance inhibits the formation of the protein into a fibril, or whether the substance causes the breakdown of the fibril, (i) inhibition of fibril formation or, (ii) the breakdown of fibrils, indicating that the substance can be used in said process. The protein is typically human islet amyloid peptide or the fibrils are typically made of human islet amyloid peptide.

The invention also provides an inhibitor identified in the use and method of the invention. Such an inhibitor may be used in any of the aspects of the invention discussed herein, or may be used in a method of treatment of the human or animal body by therapy. Thus the invention also provides a process, culture medium pre-mix, ex vivo cell, pharmaceutical composition vessel or kit of the invention wherein the inhibitor is an inhibitor that has been identified in the use or method of the invention.

A therapeutically effective number of cells of the invention may be administered to a human or animal in need of treatment. Diseases which may be treated using the cells of the invention are those in which a particular cell type is malfunctioning or has died. The condition of a patient suffering from such a disease can thus be improved.

Thus the invention provides cells of the invention for use in a method of treatment of the human or animal body by therapy, in particular for use in a method of treating diabetes. The invention also provides use of any of the cells of the invention in the manufacture of a medicament for the treatment of a disease in which a particular cell type is malfunctioning or has died, in particular for the treatment of diabetes. Thus the invention provides a method of treating a disease in which a particular cell type is malfunctioning or has died comprising administering a cell of the invention to an individual with the disease.

The term “transplantation” refers to any method of administering cells. Thus in one embodiment a surgical procedure the cells are placed in the relevant part of the body. The cells may be administered by direct injection into the relevant site. Preferably the cells are combined with a pharmaceutically acceptable carrier or diluent to produce a pharmaceutical composition. Suitable carriers and diluents include isotonic saline solutions, for example phosphate-buffered saline. The composition is typically formulated for intravenous or subcutaneous administration, or for administration by transplantation.

In one embodiment the cells are encapsulated. Generally the encapsulating material is permeable to nutrients (such as sugars or amino acids), but impermeable to immune mediators (such as antibodies or complement components) or cells.

Typically the material comprises alginate (alternating blocks of mannuronic and guluronic acid) such as in the form of barium and/or poly-L-lysine alginate. The material may comprise hollow fibres (such as acrylic, polyacrylonitrile vinyl chloride or polyethersulfone). The material may comprise hydroxyethyl-methacrylate-methyl-methacrylate, polyphosphazene or agarose.

The dose of cells which are administered to a patient will depend upon a variety of factors such as the age, weight and general condition of the patient, the disease that is being treated and the particular cells that are being administered. A suitable dose may however be from 0.1 to 100 mg/kg body weight such as 1 to 40 mg/kg body weight, or 10³ to 10⁷ cells. The routes of administration and dosages described above are intended only as a guide since a skilled physician will be able to determine readily the optimum route of administration and dosage for any particular patient and condition.

The inhibitor is administered to a patient by a route which is effective for preventing damage to transplanted cells by fibrils. Suitable routes of administration and dosages have been discussed above. Generally an effective non-toxic amount of the inhibitor is administered. The inhibitor is typically administered in the form of a pharmaceutical composition comprising the inhibitor in association with a pharmaceutically acceptable carrier or diluent. The inhibitor may be administered in any of the forms discussed above, for example with any of the pharmaceutically acceptable vehicles mentioned above. The inhibitor may be present in any of these forms when it is used in the in vitro process of the invention.

The invention is further illustrated by the following examples which should not be construed as further limiting the subject invention. The contents of all references, issued patents, and published patent applications cited throughout this application are hereby incorporated by reference.

### EXAMPLE 1

Determination of the Rate of Amyloid Fibril Formation by Thioflavine T Spectroscopy

Thioflavine T (ThT) binds to amyloid proteins in β-sheet formation, exhibiting a yellow fluorescence from tissue sections and fibrils in vitro. Detection of ThT fluorescence can be used as a sensitive assay for amyloid fibril formation under different conditions. This assay has been used in experiments to determine the effects of compounds of the invention on amyloid fibril formation.

Method

Human IAPP was dissolved in 40% trifluoroethanol and freeze-dried into conveniently-sized aliquots. IAPP was prepared immediately before the measurements by dissolving in 40% 1,1,1,3,3,3-hexafluoro-2-propanol (HFIP) in water to maintain the peptide in alpha helical conformation and soluble. A stock solution of ThT (2.5 mM) was prepared, 7.9 mg in 10 mL Tris-HCl pH 7.0 and filtered (0.22 μm). Solutions were kept in the dark until use. Fluorescence was examined at 440 nm excitation (slit 5 nm), and emission at 482 nm (slit 10 nm) with stirring. 25 ml of ThT stock (final concentration 62.5 μM) was added to peptide sample and made up to 1 mL in the cuvette. The sample was stirred for 5 min. before taking a reading. Measurements were made at an initial time point (5 min. from sample preparation), at intervals over the next 4-6 h and after overnight incubation at room temperature.

Certain compounds as disclosed herein, i.e., 3-(3-hydroxy-1-propyl)amino-1-propanesulfonic acid; DL-2-amino-5-phosphovaleric acid; 4-Phenyl-1-(3′-sulfopropyl)-1,2,3,6-tetrahydropyridine; cyclohexylsulfamic acid; O-phospho-L-serine; hexafluoroglutaric acid; 8-methoxyquinoline-5-sulfonic acid; 3-amino-2-hydroxy-1-propanesulfonic acid; and 3-dimethylamino-1-propanesulfonic acid, and 1,2,3,4-tetrahydroisoquinoline, were found to inhibit or prevent IAPP-associated fibril assembly.

### EXAMPLE 2

Circular dichroism analysis was conducted to confirm the activity of certain therapeutic compounds in preventing or inhibiting IAPP-associated fibril formation in accordance with the present disclosure by determining the presence or absence of β-sheet conformation.

The assay is conducted as follows:

Instrument and Parameters

Instrument: JASCO J-715 Spectropolarimeter

Cell/cuvette: Hellma quartz (QS) with 1.0 mm pathlength

Room temperature

Wavelength interval: 250 nm-190 nm

Resolution: 0.1 nm

Bandwidth: 1.0 nm

Response time: 1 sec

Scanning speed: 20 nm/min

Number of spectra run: 5

The assay, a co-incubation procedure, examines the ability of a compound or substance to inhibit the assembly of amyloid fibrils, e.g., to test for the presence of the amyloidotic b-sheet conformation in the presence of soluble IAPP. Samples are run in the presence and absence (i.e., water alone) of buffering agent, which is done to determine if competitive effects are seen with the ionic buffer (usually phosphate).

A. Assay in Water Only

Add components used at a molar ratio of 1:10 [peptide:compound]; add 10 μL of 10 mg/mL IAPP stock solution (final 100 μg peptide) to the aqueous solution containing compound to a final volume of 400 μl. The pH of the final assay solution is measured to ensure there is no fluctuation and the spectrum is accumulated using the parameters as shown above.

B. Assay in Phosphate Buffer

Add desired amount of compound to achieve a 1:10 molar ratio in 10 mM phosphate buffer, pH 7. Add 10 μL of 10 mg/mL IAPP stock solution (final peptide 100 μg) to the phosphate buffered solution containing the compound and bring to a final volume of 400 μL. The pH of the final assay solution is measured to ensure there is no fluctuation and the spectrum is accumulated using the parameters as shown above.

In both assays, a control sample is run with each test group. This control contains peptide only in water or buffer at a similar final volume of 400 μl. Spectra for the control are collected initially (first run) and at the end of the test (final run) to ensure that the peptide has not undergone extensive aggregation during the course of the assay. Spectra for the controls are used to compare with the measurements obtained with the treated samples.

### Co-Incubation

Make fresh 1 mg/mL stock solution of IAPP in 10 mM phosphate buffer, pH 7. Add desired amount of compound to achieve a 1:10 molar ratio in 10 mM phosphate buffer, pH 7. Incubate for 3 days at room temperature. Make up to final volume of 400 μL with 10 mM phosphate buffer, pH 7. The pH of the final assay solution is measured to ensure there is no fluctuation and the spectrum is accumulated using the parameters as shown above. A similar control is run for comparative purposes.

### Data Analysis

Plots of the spectra (control and treated) are individually assembled and the changes in ellipticity at 218 nm are examined. This minimum is directly correlated with the amount of peptide in β-sheet conformation present in the sample. Changes in either a positive or negative direction are noted and a relative value (“active” or “not active”) assigned to the compound as a measure of activity.

| Compound | Activity |
|---|---|
| 3-(3-hydroxy-1-propyl)amino-1-propanesulfonic acid | Active |
| DL-2-amino-5-phosphovaleric acid | Active |
| 1,2,3,4-tetrahydroisoquinoline, hydrochloride | Active |
| cyclohexylsulfamic acid, sodium salt | Active |
| O-phospho-L-serine | Active |
| hexafluoroglutaric acid | Active |
| 8-methoxy-5-quinolinesulfonic acid, sodium salt | Active |
| 4-Phenyl-1-(3′-sulfopropyl)-1,2,3,6-tetrahydropyridine, | Active |
| sodium salt | |
| 3-amino-2-hydroxy-1-propanesulfonic acid | Active |
| 3-dimethylamino-1-propanesulfonic acid | Active |

### EXAMPLE 3

The Synthesis of a Compound of the Invention, 4-phenyl-1-(3′-sulfopropyl)-1,2,3,6-tetrahydropyridine, Sodium Salt.

To a solution of 4-phenylpyridine (15.5 g, 0.1 mol) in acetone (100 mL) was added 1,3-propane sultone (12.2 g, 0.1 mol) at room temperature. The mixture was then heated at reflux temperature overnight. The resultant suspension was cooled to room temperature. The solid was collected by filtration and washed with acetone. To a solution of the solid (31 g) in methanol (500 mL) was added sodium borohydride (10 g, 260 mmol) portionwise, and the mixture was stirred at room temperature for 2 h. Distilled water (50 mL) was added to destroy the excess of sodium borohydride. The mixture was diluted with methanol (200 mL), and neutralized with Amberlite IR-120 ion-exchange resin (H⁺ form, 300 g). A white precipitate was formed. The precipitate and the resin were removed by filtration and treated with distilled water (400 mL) at ˜100° C. The mixture was filtered and the residual resin was washed with hot distilled water (2×200 mL). The filtrates and washings were combined and concentrated to dryness. The residue was co-evaporated with methanol (3×200 mL), and then recrystallized from ethanol-water {8:2 (v/v)} to afford 4-phenyl-1-(3′-sulfopropyl)-1,2,3,6-tetrahydropyridine as white crystals (26 g, 93%). The ¹H and ¹³C NMR spectra were in agreement with the structure.

To a solution of 4-Phenyl-1-(3′-sulfopropyl)-1,2,3,6-tetrahydropyridine (5.6 g, 20 mmol) obtained above in ethanol (180 mL) was added sodium hydroxide (1.2 g, 30 mmol). The suspension was heated at reflux temperature for 30 min. The reaction mixture was then cooled to room temperature. The first crop of product (3.9 g, 64% yield) was collected by filtration. The filtrate was concentrated to dryness, and the residue was recrystallized from ethanol to afford a second crop of product (2 g, 32% yield).

¹H NMR (400 MHz, D₂O): δ 1.85 (quintet, 2H, J 8.7, 7.7 Hz, 2H-2′), 2.39-2.45 (m, 4H, 2H-3′ and 2H-3), 2.59 (t, 2H, J 5.6 Hz, 2H-2), 2.80 (t, 2H, 17.7 Hz, 2H-1′), 3.00 (br s, 2H, 2H-6), 6.00 (br s, 1H H-5), 7.18-7.36 (m, 5H, Ar).

¹³C NMR (100.6 MHz D₂O): δ 23.90 (C-2′), 29.01 (C-3), 51.69, 51.76 (C-2, C-3′), 54.45 (C-6), 58.12 (C-1′), 123.75 (C-5), 127.31, 130.01, 131.24 (Ar), 136.89 (C-4), 142.47 (Ar).

### EXAMPLE 4

ELISA Assay for Quantitative Determination of Fibril Formation

Human IAPP (hIAPP) was synthesised by solid state synthesis (Advanced Biotechnologies, UK). Human IAPP forms fibrils immediately in aqueous media. Aliquots of IAPP in water (800 μg/ml) were stored frozen at −20° C. until use. These samples contain small numbers of fibrils visible with electron microscopy (em).

Two sets of samples containing IAPP (20 μM) with or without inhibitors (at a molar ratio of 10:1 with IAPP, (20 μM)) were prepared on day 1 in Tris buffer. One set was centrifuged, the supernatant removed and frozen immediately at −20° C. (Time zero time point). The other set was incubated overnight at room temp with shaking (time 24 h time point). On day 2 these samples were spun and supernatant removed to determine the proportion of non-fibrillar IAPP.

To assay IAPP in the supernatant both sets of samples were serially diluted in bicarbonate coating buffer, added to ELISA wells and incubated overnight. Plates were washed in phosphate buffer containing Tween (PBS/Tw)×3. Diluted antiserum raised against human IAPP was incubated on the plate for 90 minutes at 37° C. Following washing in PBS/Tw, alkaline phosphatase conjugated anti-rabbit antisera was incubated for 90 mins. Following washing in PBS/Tw, alkaline phosphate substrate was added and colour allowed to develop for 30 mins. Optical density was read at 405 nm.

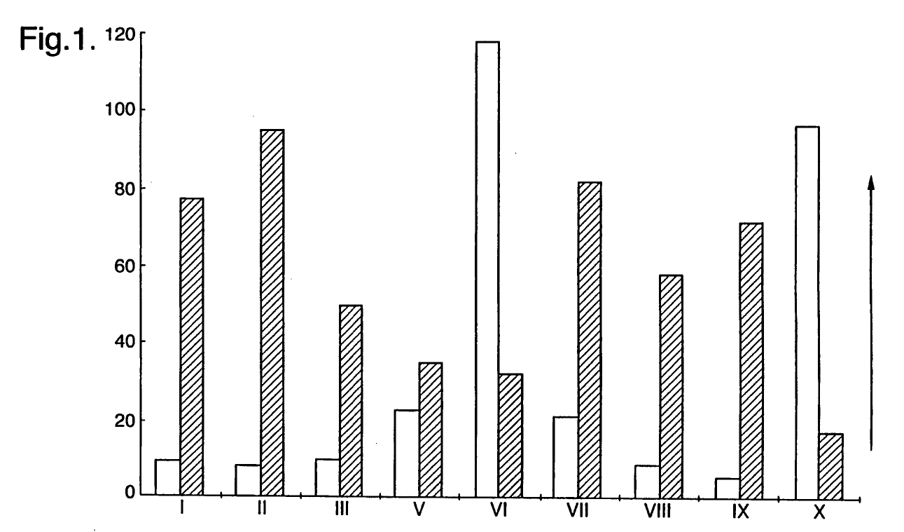

Data from all samples was calculated to determine changes of IAPP concentration in the supernatant induced by the candidate substance immediately (i.e. at time zero) or after 24 h incubation. Data in FIG. 1 is expressed as the proportion of non-fibrillar IAPP in the supernatant of test samples compared to control at both time points.

Candidate substances (i), (ii), (v), (vi), (ix), (x), the hydrochloride of (iii), and the sodium salt of (iv), (vii) and (viii) were found to increase the proportion of non-fibrillar IAPP in the supernatant. This effect was greater at time zero in samples incubated with (vi) and (x), but greater at time 24 h with all other candidate substances.

Samples of the centrifuged pellet at both time points were examined for changes in morphological characteristics of IAPP fibrils by electron microscopy.

### EXAMPLE 5

Determination of the Rate of Fibril Formation by Thioflavine T Spectroscopy

Thioflavine-T binds to proteins in β-sheet formation exhibiting a yellow fluorescence. This can be used as a sensitive assay for fibril formation under different conditions. This assay has been used in experiments to determine the effects of some candidate compounds on fibril formation.

Human IAPP was dissolved in 40% Trifuoroethanol and freeze dried into convenient sized aliquots. IAPP (1 mg/ml) was prepared immediately before the measurement by dissolving in 40% HFIP in water to maintain the peptide in alpha confirmation and soluble. A stock solution of Thioflavine T (2.5 mM) was prepared 7.9 mg in 10 ml Tris pH 7.0 and filtered (0.22 micron). Solutions were kept in dark until use. Fluorescence was examined at 440 nm excitation (slit 5 nm), and Emission at 482 nm (Slit 10 nm) with stirring. Twenty five microlitres of Th-T stock (final conc 62.5 μM) was added to peptide sample (8 μg/ml) and made up to 1 ml in the cuvette. The sample was stirred for 5 min before taking a reading. Measurements were made at an initial time points (5 mins from sample preparation) and at intervals over the next 4-6 h after overnight incubation at room temp with and without candidate compound (vi) and a compound known to accelerate fibril formation, polyvinylsulphonate.

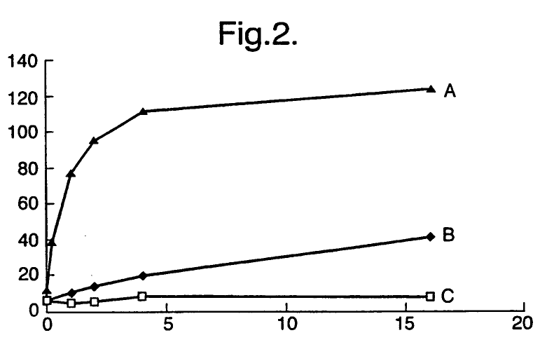

In the thioflavine T assay, polyvinylsulphonate increased the fluorescence units (FIG. 2) and compound (vi) reduced fluorescence below the level recorded for IAPP alone. This reduced level was stable for up to 15 hrs. Thus it appears that polyvinylsulphonate increased IAPP fibril formation and (vi) either reduced or prevented fibril formation over this time period.

### EXAMPLE 6

Circular Dichroic Spectroscopy

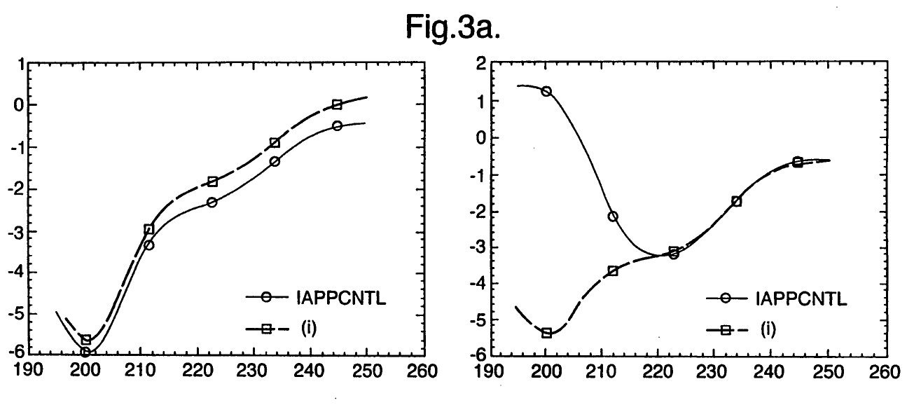

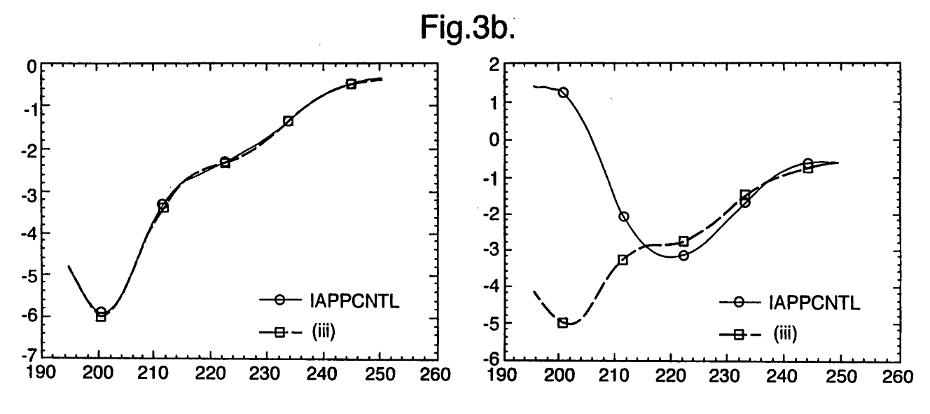

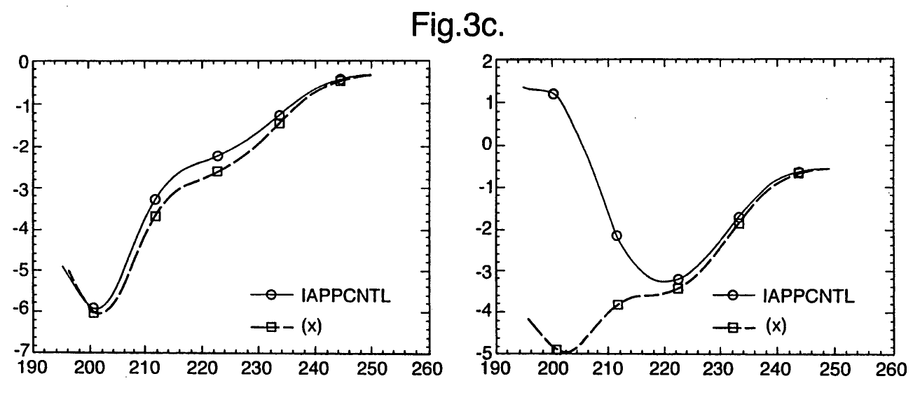

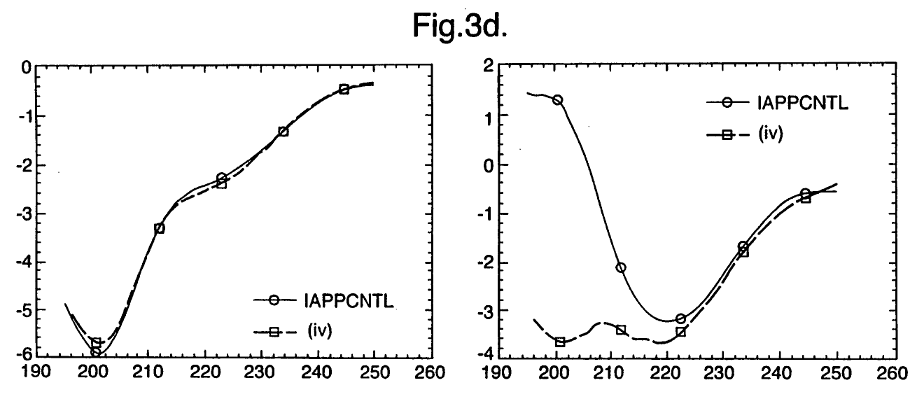

Human IAPP was prepared in 100% HFIP at 1 mg/ml filtered through a 0.31 μm filter, freeze dried in aliquots, resuspended in 20% HFIP and water, and filtered before analysis. The circular dichroic spectroscopy was performed in the same manner as described in Hubbard et al (1991) Biochem J, 275, 785-8. Samples were analysed immediately and after 24 h incubation with candidate substances i, iii, iv and x. Samples of IAPP alone (CNTL) and IAPP with candidate compound showed typical CD spectra for α-conformation at time zero. After 24 h incubation the molecular conformation of IAPP (CNTL) was converted to β-conformation. IAPP in the presence of these candidate compounds remained in α-conformation after 24 h incubation. These data indicate that with time IAPP adopts a β-conformation predicting formation of β-sheet assembly into fibrils. This is prevented by the candidate compounds. The results of the circular dichroic spectroscopy are shown in FIG. 3.

### EXAMPLE 7

Culture of Human Islets

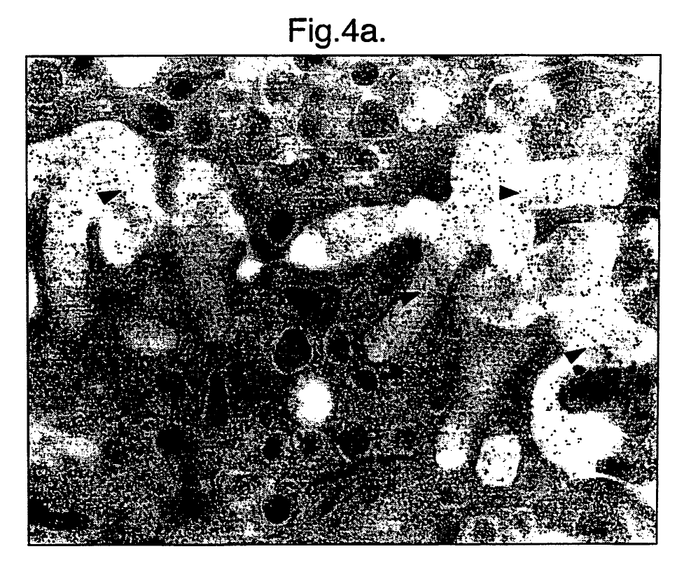

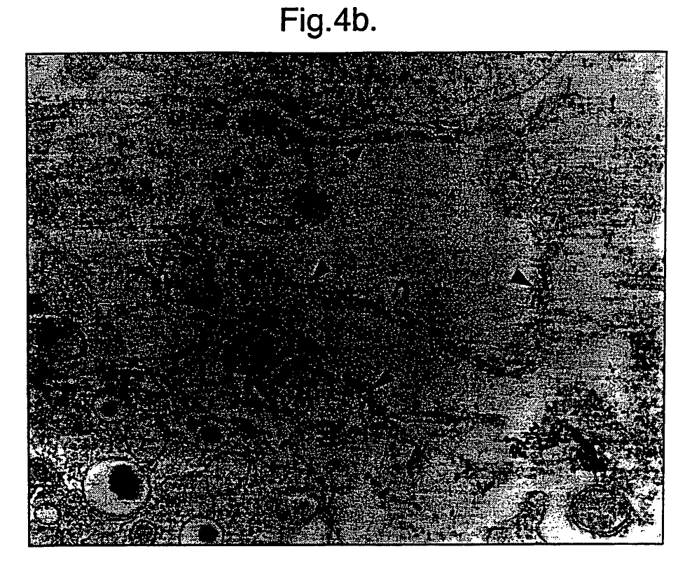

Pancreas was obtained from organ donors. Islets isolated by collagenase digestion were either handpicked from the digest or purified on a ficoll gradient. Islets were cultured, free floating in bacterial petrie dishes in different media all from Gibco Life Sciences; these included Ham F10, RPMI 1640, CMRL. The media was supplemented with glucose (5.6, 8, 11.1 or 16.7 mM), 100 u/ml benzpenicillin, 0.1 mg/ml streptomycin and 10% foetal calf serum. The media was changed every two days. Islets were cultured under sterile conditions at 37° C. in humidified air/5% Carbon dioxide for periods of 2-10 days. Fibrils formed between cultured cells were identified by electron microscopy and immunogold labelling for IAPP. FIG. 4a shows an electron micrograph of isolated human islet cells immunogold labelled for IAPP. The cells were cultured for 6 days in 8 mM glucose and RPMI.

Culture of Mouse Islets

These are used as a test system for determining the toxicity and effect of putative amyloid inhibitors in short term culture systems. Whereas normal mice can be used to determine toxicity of compounds added to the media, amyloid fibrils form only in murine islets isolated from transgenic mice expressing the gene for human IAPP (transgene incorporation was confirmed by PCR). These islets are isolated in a similar way to that of human islets and are handpicked from the digest and cultured in 16.7 mM glucose as above. Amyloid fibrils formed within 4 days of culture and can be quantified by quantitative electron microscopy. FIG. 4b shows an electron micrograph of of isolated transgenic mouse islet cells which express and secrete human IAPP. The cells were cultured for 6 days in 11.1 mM glucose and RPMI.

### EXAMPLE 9

Toxicity Study

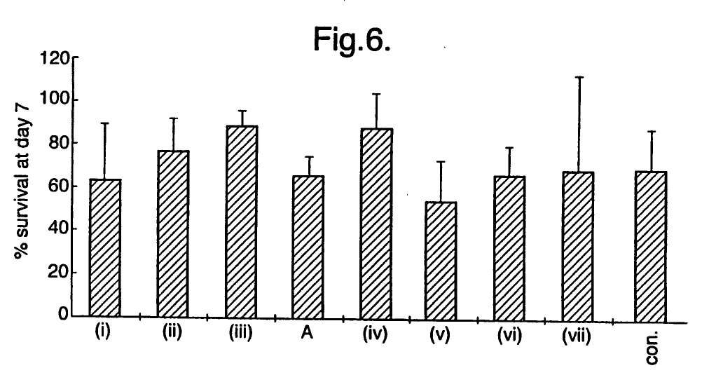

Islets from transgenic mice were cultured in the presence of 50 mM and 100 mM of the relevant compound and survival was compared with islets cultured in the absence of the compound. Mean islet survival with both concentrations of the compound is shown in FIG. 6 (mean of all experiments with both concentrations+SD). No significant differences were seen between the control (con) and test islets at either concentration with any compound tested.

### EXAMPLE 9

Election Micrograph Analysis of the Effect of Candidate Compounds

Transgenic islets cells were cultured as described above, for 7 days and the media changed every two days. At the end of the culture period the islets were fixed in 2.5% glutaraldehyde in 0.1M sodium phosphate buffer (pH 7.2) for one hour and post-fixed in 1% osmium tetroxide, and embedded in Spurr's resin (Taab Laboratories, Reading, UK) or contrast enhanced in 2% Uranyl Acetate, dehydrated in methanol and embedded in LRGold (Taab, as previous). Ultrathin sections of the islets were mounted on formvar coated nickel grids. Amyloid was identified by immunogold labelling of tissue sections with an antisera raised to human IAPP 1-37, human IAPP 17-37, rat IAPP 1-37 and goat anti-rabbit gold conjugate (10 nm diameter) either from Biocell, Cardiff, UK or Sigma or by use of protein A gold. Grids were examined in a JOEL electron microscrope at an excitation voltage of 80 kV.

Images of 3 islet sections at a low magnification were prepared. A montage of each islet was made from electron micrographs and the areas of both intra and extra cellular amyloid identified and marked. The total islet area, together with the area of intra and extra cellular amyloid was then calculated using image analysis (Kontron Image Processor). 3 separate islets were examined for each condition.

The table below shows total islet area in cultures with and without vii.

| | Extra | Total | Extra |
|---|---|---|---|
| | amyloid | islet area | amyloid % |
| | area (μm²) | (μm²) | of total |
| CONTROL | | | |
| islet 1 | 59.39 | 3271.48 | 1.8 |
| islet 2 | 281.74 | 4485.74 | 6.2 |
| islet 3 | 111.09 | 3277.28 | 3.4 |
| Average | 150.74 | 3678.17 | 4.1 |
| With vii | | | |
| islet 1 | 98.73 | 5744.23 | 1.9 |
| islet 2 | 37.9 | 7360.63 | 0.5 |
| islet 3 | 123.99 | 3616.76 | 3.4 |
| Average | 86.8 | 5407.21 | 1.6 |

### EXAMPLE 10

Morphological Characteristics of IAPP Fibrils Formed in Mixtures with Candidate Compounds.

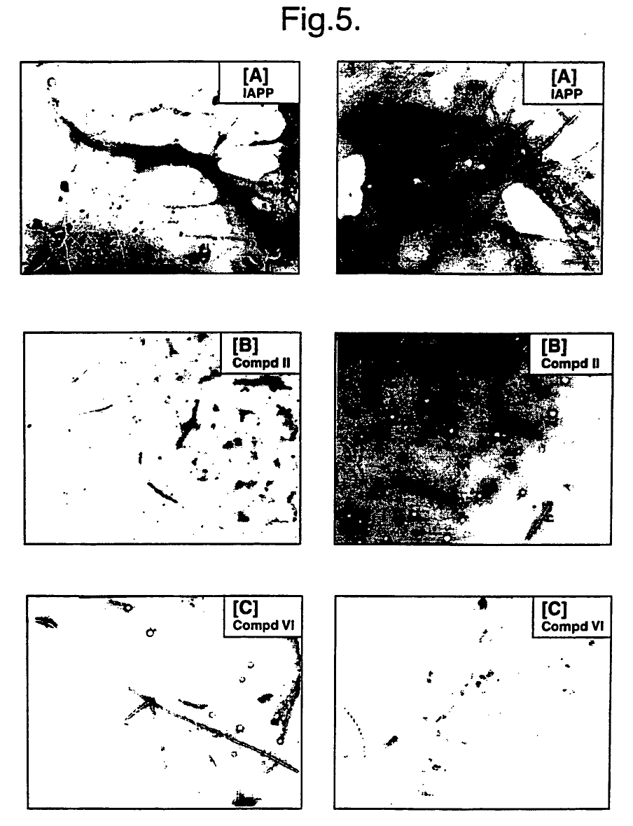

IAPP prepared as in Example 3 was incubated with candidate compounds for 48 h Samples were then examined by transmission electron microscopy for the presence of fibrils. A dense network of fibrils was visible in negatively stained preparations of IAPP (1 mg/ml) as can be seen in FIG. 5a. Long and short unbranching fibrils were present. Samples incubated with compound (ii) contained less fibrils and those present were short and not aggregated (FIG. 5b). Samples incubated with compound (vi) also contained fewer fibrils which were apparently short and long and not aggregated (FIG. 5c). These data indicate that the process of IAPP fibril formation with time has been reduced by candidate compounds (ii) and (vi).

### EOUIVALENTS

Those skilled in the art will recognize, or be able to ascertain using no more than routine experimentation, numerous equivalents to the specific procedures described herein. Such equivalents are considered to be within the scope of this invention and are covered by the following claims. The contents of all references, issued patents, and published patent applications cited throughout this application are hereby incorporated by reference.

1-21. (canceled)

22. A method for the preparation of cells suitable for treating a subject having amyloid deposits, said method comprising contacting the cells in vitro with an inhibitor of amyloid deposit formation, such that amyloid deposit formation is inhibited therein.

23. The method according to claim 22, wherein said inhibitor causes breakdown of amyloid deposits, the deposits having been formed by said cells prior to said contacting.

24. The method according to claim 22, wherein said cells are cultured in the presence of the inhibitor.

25-26. (canceled)

27. The method according to claim 22, wherein said inhibitor comprises a compound selected from the group consisting of:
(i) 3-(3-hydroxy-1-propyl)amino-1-propanesulfonic acid
HOCH₂CH₂CH₂NHCH₂CH₂CH₂SO₃H;
(ii) DL-2-amino-5-phosphovaleric acid 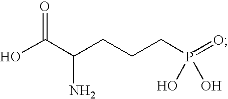
(iii) 1,2,3,4-tetrahydroisoquinoline 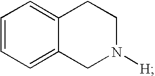
(iv) cyclohexylsulfamic acid 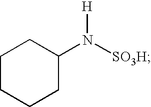
(v) O-phospho-L-serine 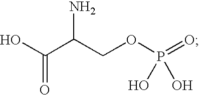
(vi) hexafluoroglutaric acid 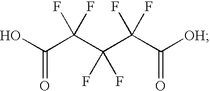
(vii) 8-methoxyquinoline-5-sulfonic acid 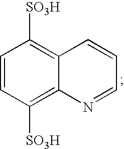
(viii) 4-phenyl-1-(3′-sulfopropyl)-1,2,3,6-tetrahydropyridine 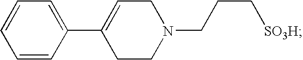
(ix) 3-amino-2-hydroxy-1-propanesulfonic acid 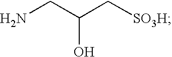
(x) 3-dimethylamino-1-propanesulfonic acid
Me₂NCH₂CH₂CH₂SO₃H;
and pharmaceutically acceptable esters, acids, and salts thereof.

28-31. (canceled)

32. A culture medium or a culture medium pre-mix comprising a compound as defined in claim 53.

33. A culture of cells in which the culture medium is as defined in claim 32.

34. A culture according to claim 33 in which the cells are islet cells.

35. Ex vivo cells prepared by the method according to claim 22.

36. Ex vivo cells according to claim 35, wherein said cells are in a preparation comprising an inhibitor, wherein said inhibitor comprises a compound selected from the group consisting of:
(i) 3-(3-hydroxy-1-propyl)amino-1-propanesulfonic acid;
(ii) DL-2-amino-5-phosphovaleric acid;
(iii) 1,2,3,4-tetrahydroisoguinoline;
(iv) cyclohexylsulfamic acid;
(v) O-phospho-L-serine;
(vi) hexafluoroglutaric acid;
(vii) 8-methoxyquinoline-5-sulfonic acid;
(viii) 4-phenyl-1-(3′-sulfopropyl)-1,2,3,6-tetrahydropyridine;
(ix) 3-amino-2-hydroxy-1-propanesulfonic acid;
(x) 3-dimethylamino-1-propanesulfonic acid;
and pharmaceutically acceptable esters, acids, and salts thereof.

37-38. (canceled)

39. Ex vivo cells according to claim 35 which are islet cells for use in a method of treating diabetes.

40. (canceled)

41. A pharmaceutical composition comprising a cell according to claim 35, and a pharmaceutically acceptable carrier or diluent.

42. Use of an inhibitor as defined in claim 53 in the manufacture of a medicament for inhibiting amyloid deposit formation by, or breaking amyloid deposits down in, a transplanted cell preparation.

43. A vessel for containing a culture of cells, wherein said vessel is coated with a compound as defined in claim 53.

44. A kit for culturing cells comprising a culture medium or culture medium pre-mix as defined in claim 32.

45. (canceled)

46. A method of identifying an inhibitor that can be used to prepare cells for transplantation in the method according to claim 22, comprising contacting a candidate substance with a mammalian cell and determining whether the candidate substance inhibits the formation of fibrils or causes the breakdown of fibrils, indicating that the substance is an inhibitor that can be used in said method.

47. A method of identifying an inhibitor that can be used to prepare cells for transplantation in the method according to claim 22, comprising contacting a candidate substance with a protein capable of forming fibrils, or with a fibril, and determining whether the substance inhibits the formation of the protein into a fibril, or whether the substance causes the breakdown of fibrils, indicating that the substance can be used in said method.

48-50. (canceled)

51. A method of inhibiting fibril formation by, or breaking fibrils down in, a transplanted cell preparation comprising administering an inhibitor as defined in claim 53 to a patient who has received a transplant of cells as defined in claim 35.

52. (canceled)

53. The method of claim 22 wherein said inhibitor of amyloid deposit formation is a compound of the formula 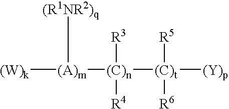
wherein k, m, t, p and q are independently 0 or 1; n is an integer from 0 to 3; C is a carbon; N is a nitrogen; W is hydrogen or an anionic group at physiological pH; Y is an anionic group at physiological pH; R¹ and R² are independently hydrogen, alkyl, an anionic group at physiological pH, or R¹ and R², taken together with the nitrogen to which they are attached, may form an unsubstituted or substituted heterocycl having from 3 to 7 atoms in the heterocyclic ring; R³ is hydrogen, halogen, thiol or hydroxyl; R⁴, R⁵, and R⁶ are independently hydrogen or halogen; and A is hydrogen or C₁ to C₆ alkyl; or a pharmaceutically acceptable ester, acid or salt thereof.

54. The method of claim 22 wherein said inhibitor of amyloid deposit formation is a compound of the formula 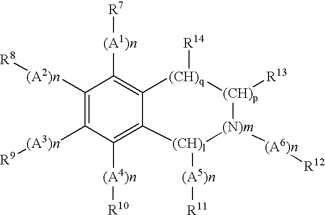
wherein C is a carbon; N is a nitrogen; H is a hydrogen; A¹, A², A³, A⁴, A⁵ and A⁶ are independently alkyl, O, S, or —NH; m and n (for each individual A group) are independently 0 or 1; p, q and 1 are independently 0, 1, or 2; R⁷, R⁸, R⁹, R¹, R¹¹, R¹², and each R¹⁴ are independently hydrogen, alkyl, alicyclyl, heterocycyl or aryl, each R¹³ is independently hydrogen, alkyl, alicyclyl, heterocycyl, aryl or an anionic group, and adjacent R groups (e.g., R⁷ and R⁸) may form an unsubstituted or substituted '0 cyclic or heterocyclic ring.
